(19) United States
(12) Patent Application Publication
(10) Pub. No.: US 2026/0335506 A1
(43) Pub. Date: Oct. 29, 2026

(54) FILTRATION ASSEMBLY AND METHODS OF USE THEREOF FOR ADIPOSE TISSUE PROCESSING

(71) Applicant: Bimini Technologies LLC, Plano, TX (US)

(72) Inventors: Elizabeth MCDERMOTT, Plano, TX (US); Horst FICHTNER, Plano, TX (US); Matthew CAMPOBASSO, Plano, TX (US); Lucas FORNACE, Plano, TX (US); Frankie NG, Plano, TX (US)

(21) Appl. No.: 19/650,651

(22) Filed: Apr. 17, 2026

Related U.S. Application Data
(60) Provisional application No. 63/794,773, filed on Apr. 25, 2025.

(51) Int. Cl. A61L 27/36; A01N 1/14

(52) U.S. Cl. CPC A61L 27/3604; A01N 1/14; A61L 2430/40

(57) ABSTRACT

The disclosure features a filtration assembly for use in the production of a dried or dehydrated adipose tissue graft, methods for making and using an adipose tissue graft, and kits containing the filtration assembly.

### BACKGROUND OF THE DISCLOSURE

The transfer of adipose tissue to various regions of the body is a relatively common cosmetic, therapeutic and structural procedure involving the harvest of adipose tissue from one location and re-implantation of the harvested and, oftentimes, processed tissue, in another location (see Coleman, S. R., “Long-term Survival of Fat Transplants: Controlled Demonstrations,” Aesth. Plast. Surg., Vol. 19, No. 5, pp. 421-425, 1995; and Coleman, S. R., “Structural Fat Grafts: The Ideal Filler?,” Clin. Plast. Surg., Vol 28, No. 1, pp. 111-119, 2001). While being largely used for repair of small cosmetic defects such as facial folds, wrinkles, pock marks, and divots, the transfer of adipose tissue has recently been used for cosmetic and/or therapeutic breast augmentation and reconstruction, and augmentation of the buttocks.

In the past, adipose tissue grafts and methods of adipose tissue transfer have been plagued with difficulties and side effects including necrosis, absorption of the implant by the body, infection, calcifications and scarring, inconsistent engraftment (Eremia, S., and Newman, N., “”Long-Term Follow-up After Autologous Fat Rafting: Analysis of Results from 116 Patients Followed at Least 12 Months After Receiving the Last of a Minimum of Two Treatments,” Dermatol. Surg., Vol. 25, No. 12, pp. 1150-1158, 2000), lack of durability, and other problems arising from lack of neovascularization and necrosis of the transplanted tissue. One of the biggest challenges in adipose tissue transfer is absorption of the implant by the body and volume retention of adipose tissue grafts following transfer. When adipose tissue is harvested or washed, the space between individual pieces of harvested adipose tissue is filled by liquid (e.g., water, blood, tumescent solution, oil). When this tissue/fluid mixture is implanted into a recipient the liquid portion is rapidly absorbed by the body resulting in loss of volume. The process by which the amount of fluid is removed from the tissue/fluid mixture is frequently referred to as “drying the adipose tissue“ or ”dehydrating the adipose tissue”. The content of red and white blood cells and the like within an adipose tissue graft can also significantly affect the volume of graft retained after graft transplantation, due to induction or exacerbation of an inflammatory response. Another aspect of tissue retention relates to the amount of lipid within the adipose tissue graft. It is understood that the presence of free lipid (meaning lipids released from dead or damaged adipocytes; also referred to as oil) in adipose tissue grafts can result in induction or exacerbation of an inflammatory response with substantial phagocytic activity and consequent loss of graft volume.

It is also known that mixing unprocessed adipose tissue with a concentrated population of adipose-derived regenerative cells overcomes many of the problems associated with adipose tissue grafts and adipose tissue transfer, such as those described above. Specifically, supplementing unprocessed adipose tissue with concentrated populations of adipose-derived cells comprising adipose-derived stem cells increases the weight, vascularization, and retention of fat grafts (see, e.g., U.S. Pat.

No. 7,390,484 and US 2005/0025755, herein expressly incorporated by reference in their entireties). Adipose tissue fragments supplemented, or mixed, with a concentrated population of cells including adipose-derived stem cells exhibit improved neoangiogeneis and perfusion in grafts when compared to unsupplemented grafts of adipose tissue alone in animal models. Further, adipose tissue grafts supplemented with adipose-derived regenerative cells that comprise adipose-derived stem cells show increased graft retention and weight over time when compared to unsupplemented grafts (see, e.g., US 2005/0025755). Further, the processing of adipose tissue in a closed, sterile fluid pathway greatly reduces the chance of infection. The improvement in autologous transfer of adipose tissue seen in the animal models described above has also been replicated in human clinical studies. Nevertheless, the isolation and purification of concentrated populations of adipose-derived regenerative cells comprising adipose-derived stem cells (ADSCs), usually involves a series of washing, digestion, filtration and/or centrifugation steps, which can reduce the yield of viable cells, require mechanical equipment and specialized clinicians, and/or can compromise the quality, appearance, longevity, hydration, and/or efficacy of the graft.

Therefore, there is a need for improved devices and methods for the preparation of adipose tissue grafts.

### SUMMARY OF THE DISCLOSURE

The present disclosure features a filtration assembly, kits, and method of use for the production of adipose tissue grafts (e.g., “fat grafts”) or adipose tissue implants. The filtration assemblies disclosed herein can be used to prepare adipose tissue grafts which have reduced levels of fluid, blood cells, and free lipid or oil content as compared to grafts prepared using conventional techniques or prior known filtration devices.

In a first aspect, the filtration assembly for processing an adipose tissue includes: a) a first outer shell and a second outer shell that are sealingly joined to form a compartment within the assembly, wherein each of the first outer shell and/or the second outer shell including a surface; b) a folded filter formed by a first segment folded onto a second segment that is located between the first outer shell and the second outer shell, wherein the folded filter forms a first subsystem within an interior of the folded filter and a second subsystem located between the first outer shell and the second outer shell on an exterior of the folded filter, wherein the folded filter further includes a first plurality of pores, the second subsystem optionally further comprises a spacer filter; and the second subsystem is in fluid communication with at least one channel; c) one or more ports in fluidic communication with the first subsystem; and d) one or more exit ports in fluid communication with the second subsystem and/or the at least one channel, wherein the one or more ports in communication with the first subsystem are configured to receive a material including the adipose tissue and to deliver the adipose tissue to the first subsystem, the first plurality of pores are sized to retain adipose cells present in the adipose tissue, and the at least one channel in the second subsystem is in fluid communication with the one or more exit ports configured to discharge the non-adipose tissue from the filtration assembly.

In some embodiments, the first outer shell and the second outer shell of the filtration assembly are joined by a double heat seal. In some embodiments, the first segment and the second segment of the folded filter, of the filtration assembly, are joined by a double heat seal, thereby forming the interior of the folded filter. In some embodiments, the exterior of the folded filter is joined to the first outer shell and the second outer shell by a double heat seal.

In some embodiments, the one or more fluids includes a tumescent fluid and/or one or more wash solutions.

In some embodiments, the filtration assembly is configured to prepare an adipose tissue comprising mature adipocytes, regenerative cells, stem cells, progenitor cells, and/or connective tissue. In some embodiments, the first plurality of pores of the filtration assembly is sized to allow passage of non-adipose tissue including one or more fluids and red blood cells present in the adipose tissue into the second subsystem, thereby removing fluid from the adipose tissue and producing a dehydrated adipose tissue. In some embodiments, the first plurality of pores have a pore size of from about 30 μm to about 2000 μm. In some embodiments, the first plurality of pores have a pore size of from about 30 μm to about 200 μm.

In some embodiments, the filtration assembly further includes the spacer filter, wherein the spacer filter has a second plurality of pores with a pore size of from about 300 μm to about 3000 μm. In some embodiments, the pore size of the second plurality of pores of the spacer filter is about 800 μm. In some embodiments, the spacer filter is present in the filtration assembly and is free floating within the second subsystem. In some embodiments, the spacer filter includes one or more ribs. In some embodiments, the spacer filter includes one or more struts.

In some embodiments, the filtration assembly further includes at least one channel that is formed by the spacer filter and the first outer shell and/or the second outer shell of the assembly. In some embodiments, the at least one channel is formed by a texture on a surface of the first outer shell and/or the second outer shell of the assembly. In some embodiments, the texture on the surface of the first and/or second outer shell of the assembly includes bumps, ridges, or grooves. In some embodiments, the at least one channel is configured to promote passage of the one or more fluids and red blood cells to the one or more exit ports.

In some embodiments, the one or more ports in fluidic communication with the first subsystem includes a first port. In some embodiments, the first port is an injection port configured to releasably connect with an adapter, wherein the adapter is configured to releasably connect with a syringe barrel. In some embodiments, the syringe barrel is a catheter syringe barrel, wherein, optionally, the syringe has a volume of about 15 mL to about 100 mL. In some embodiments, the first port is a tissue access port assembly including a deformable plastic valve configured to seal the tissue access port assembly; wherein the deformable plastic valve is configured to facilitate delivery of material (e.g., a lipoaspirate) to the first subsystem or removal of material from the first subsystem upon insertion of a cannula therethrough. In some embodiments, the first port comprises a deformable plastic valve. In some embodiments, the first port is configured to be attached to a cannula.

In some embodiments, a first exit port of the one or more exit ports is in communication with the second subsystem and is configured to discharge non-adipose tissue from the first exit port, wherein the first exit port is configured to inhibit the material from entering the first exit port. In some embodiments, the filtration assembly further includes a second port of the one or more ports in fluidic communication with the first subsystem. In some embodiments, the one or more exit ports is connected to a branching connector.

In a second aspect, the disclosure features a method producing an adipose tissue graft including: (a) introducing an adipose tissue into a first subsystem of a filtration assembly of the first aspect via the one or more ports in fluidic communication with the first subsystem; and (b) removing a flow-through fluid that passes from the first subsystem to a second subsystem of the filtration assembly via the one or more exit ports, wherein the flow-through fluid includes water, blood, free lipid, or any combination thereof, thereby producing the adipose tissue graft.

In some embodiments, the adipose tissue is obtained from a subject prior to step (a). In some embodiments, the adipose tissue is a lipoaspirate, an unprocessed portion of adipose tissue, partially disaggregated adipose tissue, or fully disaggregated adipose tissue.

In some embodiments, prior to step (b) of the method, the filtration assembly is agitated to produce a partially or fully disaggregated adipose tissue within the first subsystem.

In some embodiments, the method further includes: (c) introducing a wash solution to the first subsystem via the one or more ports in fluidic communication with the first subsystem, thereby rinsing the adipose tissue; and/or (d) removing a waste fluid that passes from the first subsystem to a second subsystem of the filtration assembly via the one or more exit ports, wherein the waste fluid includes water, the wash solution, blood, free lipid, or any combination thereof, thereby producing a dehydrated adipose tissue graft.

In some embodiments, the wash solution includes Lactated Ringer's solution, Hartmann's Solution, and/or saline. In some embodiments, during step (c) of the method, the filtration assembly is agitated to produce a partially or fully disaggregated adipose tissue within the first subsystem. In some embodiments, the method further includes repetition of steps (c)-(d) at least once.

In some embodiments, the dehydrated adipose tissue graft produced by the method has a liquid content that is less than about 50% of the liquid content of the adipose tissue of step (a). In some embodiments, the dehydrated adipose tissue graft produced by the method has a liquid content that is less than about 33.3% of the liquid content of the adipose tissue of step (a). In some embodiments, the dehydrated adipose tissue graft produced by the method has a liquid content that is less than about 25% of the liquid content of the adipose tissue of step (a).

In a third aspect, the disclosure features a method of treating a subject comprising administering an adipose tissue graft prepared according to the method of the second aspect to the subject. In some embodiments, the adipose tissue graft is a dehydrated adipose tissue graft. In some embodiments, the adipose tissue graft administered to the subject is aseptically removed from the filtration assembly prior to administration. In some embodiments, the adipose tissue graft is administered to an area in the body of the subject in need of treatment. In some embodiments, the adipose tissue graft is administered to the subject during reconstructive surgery or augmentation. In some embodiments, the reconstructive surgery or augmentation is breast reconstructive surgery or augmentation.

In a fourth aspect, the disclosure features a kit including a filtration assembly of the first aspect and optionally, one or more of: (i) a filtration assembly holder; (ii) a waste container; (iii) a wash solution holding container; and (iv) a squeegee.

### Definitions

To facilitate an understanding of this disclosure, a number of terms are defined below. Terms defined herein have meanings as commonly understood by a person of ordinary skill in the areas relevant to the disclosure. Terms such as “a,” “an,” and “the” are not intended to refer to only a singular entity (unless context reveals otherwise) but include the general class of which a specific example can be used for illustration.

As used herein, the term “about” refers to a value that is within 10% above or below (±10%) the value being described.

As used herein, “adipose-derived regenerative cell” refers to any cells obtained from adipose tissue which cause or contribute to complete or partial regeneration, restoration, or substitution of structure or function of an organ, tissue, or physiologic unit or system to thereby provide a therapeutic, structural, or cosmetic benefit. Examples of regenerative cells include: adipose-derived stem cells (“ADSCs”), endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes.

As used herein, the terms “administering,” “introducing,” “delivering,” “placement,” and “transplanting” are used interchangeably herein and refer to the placement of the compositions disclosed herein, e.g., supplemented fat grafts, into a subject by a method or route which results in at least partial localization of the transplant or fat graft at a desired site. In some cases, the supplemented adipose tissue graft (e.g., the adipose graft supplemented with adipose-derived regenerative cells) can be administered to the subject without being removed from the system or exposed to the external environment of the system or device in which it was generated prior to administration.

As used herein, the terms “dried adipose tissue” or “dehydrated adipose tissue” refer to a unit of adipose tissue having a lower content of liquid, e.g., water or other liquid (e.g., tumescent fluid), present therein as compared to unprocessed adipose tissue from the same site and/or same subject (e.g., an equivalent unit (w/w) of adipose tissue taken from the same site and same subject as the adipose tissue that was dried).

As used herein, “endothelial progenitor cell” refers to a multipotent or unipotent cell with the potential to differentiate into vascular endothelial cells.

As used herein, “supplemented adipose tissue grafts” refer to adipose tissue grafts that have been combined with adipose-derived regenerative cells or adipose-derived stem cells. The added adipose-derived regenerative cells or adipose-derived stem cells may be obtained from the same subject. Alternatively, the added adipose-derived regenerative cells or adipose derived stem cells can be obtained from a different subject.

As used herein, the term “equivalent unit” refers to an equivalent volume or weight of adipose tissue obtained from a subject. For example, an equivalent unit can mean an equivalent volume (or weight) of adipose tissue obtained from a subject. An equivalent unit can be, e.g., an equivalent volume (or weight) obtained from the same site (e.g., buttocks, abdomen, thigh, back, or the like) from the same or a different subject.

As used herein, the term “lipoaspirate” refers to a portion of material removed from a subject during liposuction. For example, a lipoaspirate may contain cellular matrix including adipocytes, adipose derived stem cells (ADSC), preadipocytes, pericytes, endothelial cells, and hematopoietic lineage cells.

Lipoaspirate may further contain water, saline, Lactated Ringer's solution, and other components of tumescent solution.

As used herein, the term “precursor cell” refers to a unipotent regenerative cell with the potential to differentiate into a single cell type. Precursor cells and their progeny may retain extensive proliferative capacity, e.g., lymphocytes and endothelial cells, which can proliferate under appropriate conditions.

As used herein, the term “progenitor cell” refers to a multipotent regenerative cell with the potential to differentiate into more than one cell type. “Progenitor cell”, as used herein, also refers to a unipotent regenerative cell with the potential to differentiate into only a single cell type, which performs one or more specific functions and has limited or no ability to self-renew.

As used herein, the term “regenerative cell composition” refers to a cell solution that comprises a population of regenerative cells, e.g., stem cells. A regenerative cell composition can include multiple different types of regenerative cells, including ADSCs, endothelial cells, endothelial precursor cells, endothelial progenitor cells, macrophages, fibroblasts, pericytes, smooth muscle cells, preadipocytes, differentiated or de-differentiated adipocytes, keratinocytes, unipotent and multipotent progenitor and precursor cells (and their progeny), and lymphocytes. Regenerative cell compositions may include only one, only two, only three, only four, or more, different types of regenerative cells. Regenerative cell compositions can also contain one or more contaminants, such as collagen, which may be present in the tissue fragments.

As used herein, the term “subject” includes warm-blooded animals, such as a mammal, for example, a primate (e.g., a human). In a particular embodiment, the subject is a human.

As used herein, the term “unit of adipose tissue” refers to a discrete or measurable amount of adipose tissue, which can be measured by determining the weight and/or volume of the unit. A unit of adipose tissue may refer to the entire amount of adipose tissue removed from a subject, or an amount that is less than the entire amount of adipose tissue removed from a subject. Thus, a unit of adipose tissue may be combined with another unit of adipose tissue to form a unit of adipose tissue that has a weight or volume that is the sum of the individual units.

As used herein, the term “unprocessed adipose tissue” refers to adipose tissue that has not been disaggregated, e.g., by subjecting the tissue to mechanical and/or enzymatic disaggregation. As such, unprocessed tissue contains intact tissue fragments, that include connective tissue bound to adipose-derived regenerative cells.

As used herein, the term “partially disaggregated adipose tissue” refers to adipose tissue in which processing, e.g., by mechanical and/or enzymatic disaggregation, has been initiated but for which an amount of the adipose tissue obtained from a subject has not been completely disaggregated. As such, partially disaggregated adipose tissue may contain intact tissue fragments, e.g., tissue fragments that include connective tissue bound to adipose-derived regenerative cells.

As used herein, the term “fully disaggregated adipose tissue” refers to adipose tissue that has been fully disaggregated, e.g., by subjecting the tissue to mechanical and/or enzymatic disaggregation. Fully disaggregated adipose tissue does not contain intact tissue fragments, including connective tissue bound to adipose-derived regenerative cells.

As used herein, any values provided in a range of values include both the upper and lower bounds, and any values contained within the upper and lower bounds.

### BRIEF DESCRIPTION OF THE DRAWINGS

FIG. 1A is an image showing folded filter 1000 of a filtration assembly of the disclosure. Folded filter 1000 includes first filter segment 1100, second filter segment 1200, and first plurality of pores 1400. Folded filter 1000 further has folding line of symmetry 1300 around which first filter segment 1100 and second filter segment 1200 is folded to produce folded filter 1000. Second filter segment 1500 further has the location of double heat seal 1500 shown.

FIG. 1B are images showing front and side views of folded filter 1000, showing the orientation of first filter segment 1100 and second filter segment 1200 after folding.

FIG. 2A is an image showing an assembled filtration assembly 2000.

FIG. 2B is an exploded view of filtration assembly 2000 of FIG. 2A, including first outer shell 2100, folded filter 1000, spacer filter 2200 including second plurality of pores 2220, and second outer shell 2300. Filtration assembly 2000 further includes exit port 3000, and ports 4000 and port 5000. Exit port 3000 is shown with drainage tube 3100, which is not integral to filtration assembly 2000 and is inserted into exit port 3000 as part of lid 9100, which is configured to engage with filtration assembly 2000 (see FIG. 8). Filtration assembly 2000 also includes double heat seal locations 1500, 2400, 2500, and 2600.

FIG. 3A is an image showing a cross-sectional view taken along section line A-A of FIG. 2A, displaying the spatial configuration of components of filtration assembly 2000, including folded filter 1000 with first plurality of pores 1400, first outer shell 2100, second outer shell 2300, spacer filter 2200, exit port 3000 including an inserted drainage tube 3100, which is part of lid 9100, port 5000, first subsystem 6100, and second subsystem 6200.

FIG. 3B is an image showing a cross-sectional view taken along section line B-B of FIG. 2A, displaying the spatial configuration of components of filtration assembly 2000, including folded filter 1000 with first plurality of pores 1400, first outer shell 2100, second outer shell 2300, spacer filter 2200, exit port 3000, ports 4000 and 5000, first subsystem 6100, and second subsystem 6200.

FIG. 4A is an image showing the potential flow path of fluids and red blood cells that enter first subsystem 6100, within filtration assembly 2000, via port 5000, flow through folded filter 1000 into second subsystem 6200, and out through exit port 3000 via drainage tube 3100.

FIG. 4B is an image showing adipose tissue 7000 being captured inside first subsystem 6100, formed by folded filter 1000, after entering filtration assembly 2000 via port 5000.

FIG. 5 is an image showing an example of a surface texture on the inward facing surface of first outer shell 2100 or the second outer shell 2300, which include groves 7100 and bumps 7200.

FIG. 6A is an image showing a tissue access port assembly 8100.

FIG. 6B is an image showing a cross-section view taken along section line A-A of FIG. 6A displaying the internal configuration of tissue access port assembly 8100 including large bore cylindrical opening 8110, deformable plastic valve 8120, narrow opening 8130, cap 8140, and internal cavity 8150.

FIG. 7A is an image showing filtration assembly holder 9000 including lid 9100, body 9200, and base 9300.

FIG. 7B is an image showing filtration assembly holder 9000 including lid 9100, body 9200, and base 9300 with filtration assembly 2000 including exit port 3000, ports 4000 and 5000 including tissue access port assembly 8100, affixed to the outside of the filtration assembly holder 9000.

FIG. 8 is an image showing lid 9100 of filtration assembly holder 9000 including tissue inlet 9110, bypass valve 9120, bypass channel 9125, drainage channel 9130, vacuum connector 9140, and waste drainage tube 9150. Lid 9100 is shown connected to filtration assembly 2000, as it would be while filtration assembly 2000 is located inside filtration assembly holder 9000. Filtration assembly 2000 is connected to lid 9100 via access port assembly 8100 that is connected to port 5000. Lid 9100 is further attached to filtration assembly 2000 via exit port 3000 attached to drainage channel 9130.

FIG. 9A is an image showing filtration assembly 2000 connected to lid 9100. Drainage tube 3100 from lid 9100 is shown inserted through exit port 3000 and into filtration assembly 2000. Also shown is a directional arrow, which indicates the flow of lipoaspirate, including adipose tissue 7000, from a subject into filtration assembly 2000. Also identified are folded filter 1000 with first plurality of pores 1400, first outer shell 2100, second outer shell 2300, spacer filter 2200, exit port 3000 with drainage tube 3100 from lid 9100, port 5000, first subsystem 6100, and second subsystem 6200, within filtration assembly holder 9000. Filtration assembly 2000 is situated within filtration assembly chamber 9210 of body 9200 and connected to lid 9100 (e.g., see FIG. 8). Adipose tissue 7000 is shown entering tissue inlet 9110 of lid 9100 and entering first subsystem 6100 formed by folded filter 1000, via tissue access port assembly 8100 and port 5000.

FIG. 9B is an image showing bypass valve 9120 releasing material (e.g., adipose tissue 7000) into bypass channel 9125 which then flows into waste chamber 9220 of body 9200 when the pressure within filtration assembly 2000 exceeds a threshold.

FIG. 10A is an image showing the process of removing waste material 7010, which has entered first subsystem 6100 of filtration assembly 2000 via tissue inlet 9110 of lid 9100 and further through tissue access port assembly 8100 and port 5000, and has flowed through first plurality of pores 1400 of folded filter 1000 into second subsystem 6200. The image further shows directional arrows showing the flow of waste material 7010 from second subsystem 6200 into waste chamber 9220 of body 9200 via drainage tube 3100 and out exit port 3000 into drainage channel 9130, which is connected to exit port 3000 with the assistance of a vacuum connected to waste chamber 9220 via vacuum connector 9140 and waste drainage tube 9150.

FIG. 10B is an image showing the removal of waste material 7010 via waste drainage tube 9150 and vacuum connector 9140 after waste chamber 9220 of body 9200 has been filled with waste material 7010 to reach waste drainage tube 9150, through the process described in FIG. 10A.

### DETAILED DESCRIPTION

The present disclosure features a filtration assembly, kits, and method of use for the production of adipose tissue grafts (e.g., “fat grafts”) or adipose tissue implants, either alone or supplemented with adipose-derived regenerative cells (e.g., a cell population that comprises adipose-derived stem cells, endothelial cells, and/or endothelial progenitor cells). The embodiments disclosed herein are based, in part, on the discovery of a filtration assembly that can be used for the rapid preparation of adipose tissue grafts and implants with greater efficiency and reduced loss of adipose tissue suitable for engraftment.

The methods can also be used for the preparation of grafts and implants enriched with a population of adipose-derived regenerative cells (e.g., a cell population comprising adipose derived stem cells). The methods include, for example, the use of gravity flow and, if desired, the absence of centrifugation, high pressure, and/or vacuum filtration. As discussed herein, the adipose tissue grafts prepared using the filtration assembly disclosed herein have reduced levels of fluid, blood cells, and free lipid or oil content as compared to grafts prepared using conventional techniques or prior known filtration devices. Notably, the adipose tissue grafts prepared using the filtration assembly disclosed herein need not be subjected to strong mechanical forces, which may lead to decreased cell viability and reduced retention of the adipose tissue graft. Using the filtration assembly disclosed herein, one can obtain a more predictable, consistent, and stable adipose-tissue implant.

The filtration assembly of the disclosure includes a first outer shell, a second outer shell, and a folded filter including a first plurality of pores. The first outer shell, second outer shell, and the folded filter are joined together to form the system, wherein the folded filter is internal to the first and second outer shells. The filtration assembly further includes a first subsystem formed by the folded filter within the assembly and a second subsystem that includes the remainder of the internal volume not occupied by the first subsystem within the assembly. The assembly further includes one or more (e.g., one, two, or three) exit ports in fluidic communication with the second subsystem and one or more (e.g., one, two, or three) ports in fluidic communication with the first subsystem. The first subsystem, as defined by the folded filter, is designed to receive lipoaspirate, via a port in fluidic communication with the first subsystem, filter out (e.g., capture) material of interest (e.g., adipose tissue e.g., adipocytes and extracellular matrix) and pass through (e.g., through the first plurality of pores of the folded filter) one or more fluids (e.g., plasma, serum, wash solution(s), or oils) and/or non-target cells (e.g., red blood cells) to the second subsystem. The second subsystem further include a spacer filter made from a material that is lipid-wicking and/or fluid-wicking (e.g., a meshwork design that draws fluid from the first subsystem) and the one or more exit ports may further be connected to a drainage tube to facilitate the removal of waste material from the second subsystem. The internal surfaces of the first and second outer shells may also include bumps, ridges, and groves to facilitate the removal of waste material out of second subsystem via drainage tube and/or one or more exit ports.

The captured material (e.g., adipose cells and/or adipose tissue) may further be washed by introducing a washing solution via one or more ports in fluidic communication with the first subsystem.

The wash solution and any fluids and/or materials (e.g., blood and/or wash solution) smaller than the first plurality of pores (e.g., about 30 μm to about 2000 μm) on the folded filter will flow into the second subsystem and be removed via the drainage tube and/or the one or more exit ports. The step of washing the trapped adipose tissue further purifies (e.g., removes contaminates) and dries (e.g., by removing about 50% to about 99% (e.g., about 55%, about 60%, about 65%, about 70%, about 80%, about 90%, about 95%, or about 97%) of the liquid (e.g., fluid present in the original injected tissue (e.g., water, wash solution, serum, and/or plasma)) of the original injected tissue). The adipose tissue retained within the first subsystem may then be removed using one or more of the ports in fluidic communication with the first subsystem (e.g., by maneuvering the assembly to orient the one or more ports in a downward facing direction and using gravity flow and/or by using a squeegee to assist in removal from the assembly, such as by gently mechanically pushing the tissue out of the one or more ports in fluidic communication with the first subsystem).

Reference will now be made in detail to particular embodiments of the disclosure, examples of which are illustrated in the accompanying drawings. Wherever possible, the same or similar reference numbers are used in the drawings and the description to refer to the same or like parts. It should be noted that the drawings are in simplified form and are the components shown are not drawn to precise scale. In reference to the disclosure herein, for purposes of convenience only, directional terms, such as top, bottom, left, right, up, down, over, above, below, beneath, rear, and front, are used with respect to the accompanying drawings. Such directional terms should not be construed to limit the scope of the disclosure in any manner.

Although the disclosure herein refers to certain illustrated embodiments, it is to be understood that these embodiments are presented by way of example and not by way of limitation. The intent of the following detailed description, although discussing particular embodiments, is to be construed to cover all modifications, alternatives, and equivalents of the embodiments as may fall within the spirit and scope as defined by the appended claims. Aspects of the present disclosure may be practiced in conjunction with various cell or tissue separation techniques that are conventionally used in the art, and only so much of the commonly practiced process steps are included herein as are necessary to provide an understanding of the present disclosure.

### Adipose Tissue Filtration Assembly

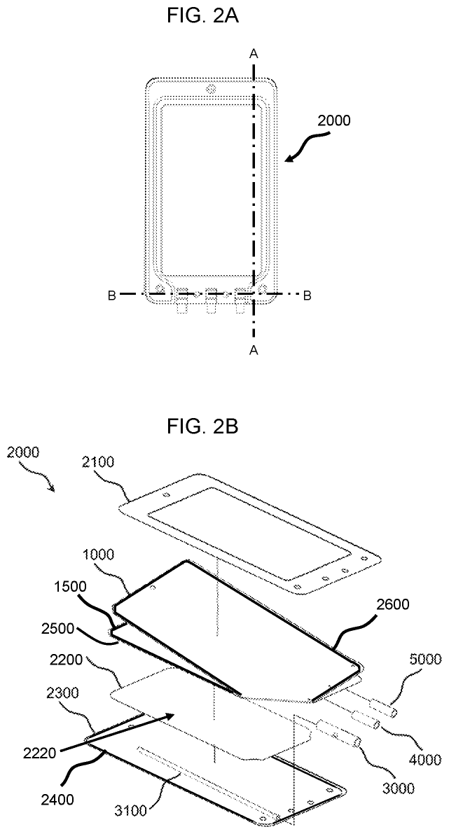

The adipose tissue filtration assembly of the disclosure also features filtration assembly 2000. Filtration assembly 2000 may be used for making dried or dehydrated adipose tissue which may be used to produce supplemented adipose tissue grafts. Filtration assembly 2000 includes first outer shell 2100, folded filter 1000, optional spacer filter 2200, and second outer shell 2300. Filtration assembly 2000 further include exit port 3000, and ports 4000 and/or 5000. Exit port 3000 may be connected to drainage tube 3100 (FIG. 2).

In some embodiments, first outer shell 2100 and second outer shell 2300 are joined by double heat seal 2400 around the entire periphery, excluding locations of exit port 3000 and ports 4000 and 5000. In some embodiments, first outer shell 2100 is joined to the outer surface of folded filter 1000 by double heat seal 2500. In some embodiments, second outer shell 2300 is joined to the outer surface of folded filter 1000 by double heat seal 2600.

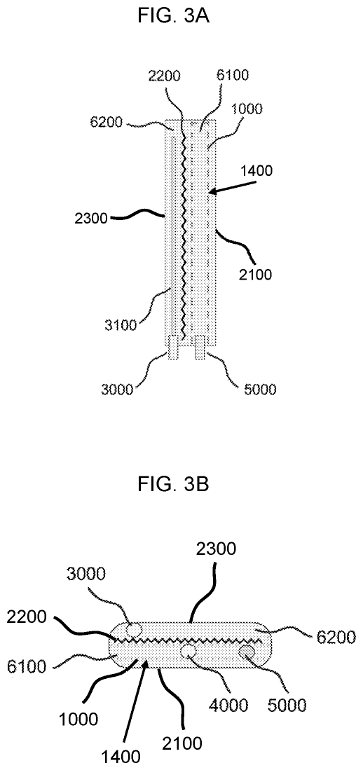

In some embodiments, folded filter 1000 forms a first subsystem 6100 of filtration assembly 2000 (FIG. 3). In some embodiments, first subsystem 6100 further includes at least one of ports 4000 or 5000. In some embodiments, at least one of ports 4000 or 5000 is an input port configured to receive an adipose tissue sample into first subsystem 6100 of filtration assembly 2000.

In some embodiments, second subsystem 6200 of filtration assembly 2000 is located between first outer shell 2100, second outer shell 2300, and folded filter 1000 (FIG. 3). In some embodiments, second subsystem 6200 further includes at least one of exit port 3000 and/or ports 4000 and/or 5000, and/or at least one of ports 4000 and 5000 is an exit port. In some embodiments, second subsystem 6200 further includes spacer filter 2200. In some embodiments, spacer filter 2200 is free floating within second subsystem 6200.

In some embodiments, second subsystem 6200 comprises multiple chambers. In some embodiments, the multiple chambers of second subsystem 6200 are not in fluidic communication with one another. In some embodiments, the multiple chambers of second subsystem 6200 are each in fluidic communication with first subsystem 6100. In some embodiments, the multiple chambers of second subsystem 6200 are in fluidic communications with one another. In some embodiments, the multiple chambers of second subsystem 6200 are in fluidic communications with one another and with first subsystem 6100. In some embodiments, each of the multiple chambers of second subsystem 6200 further include an exit port 3000. In some embodiments, the multiple chambers of second subsystem 6200 are in fluidic communication with one another, and with a single exit port 3000.

In some embodiments, spacer filter 2200 includes second plurality of pores 2220. In some embodiments, second plurality of pores 2220 may have a diameter of about 300 μm to about 3000 μm (e.g., about 300 μm to about 3000 μm, about 350 μm to about 3000 μm, about 400 μm to about 3000 μm, about 450 μm to about 3000 μm, about 500 μm to about 3000 μm, about 600 μm to about 3000 μm, about 700 μm to about 3000 μm, about 800 μm to about 3000 μm, about 900 μm to about 3000 μm, about 1000 μm to about 3000 μm, about 1250 μm to about 3000 μm, about 1500 μm to about 3000 μm, about 1750 μm to about 3000 μm, about 2000 μm to about 3000 μm, or about 2500 μm to about 3000, e.g., about 300 μm, about 350 μm, about 400 μm, about 450 μm, about 500 μm, about 600 μm, about 700 μm, about 800 μm, about 900 μm, about 1000 μm, about 1250 μm, about 1500 μm, about 1750 μm, about 2000 μm, or about 2500 μm). In some embodiments, second plurality of pores 2220 may have a diameter of about 500 μm to about 1000 μm (e.g., about 500 μm to about 1000 μm, about 550 μm to about 1000 μm, about 600 μm to about 1000 μm, about 650 μm to about 1000 μm, about 700 μm to about 1000 μm, about 800 μm to about 1000 μm, about 850 μm to about 1000 μm, about 900 μm to about 1000 μm, about 950 μm to about 1000 μm, about 550 μm to about 950 μm, about 600 μm to about 900 μm, about 650 μm to about 850 μm, or about 700 μm to about 800, e.g., about 500 μm, about 550 μm, about 600 μm, about 650 μm, about 700 μm, about 800 μm, about 850 μm, about 900 μm, about 950 μm, or about 1000 μm). In some embodiments, spacer filter 2200 includes a texture. In some embodiments the texture on spacer filter 2200 may include one or more ribs or one or more struts.

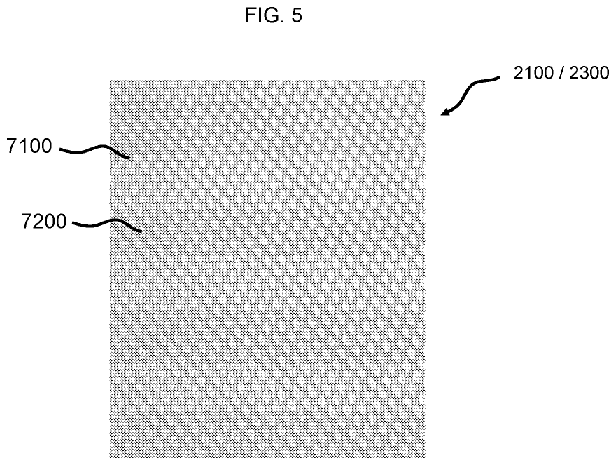

In some embodiments, first outer shell 2100 and/or second outer shell 2300 of filtration assembly 2000 may have a texture (e.g., FIG. 5) on the inner surface of outer shell 2100 and/or 2300. In some embodiments, the texture on first outer shell 2100 and/or second outer shell 2300 may include bumps, ridges, or grooves.

In some embodiments, second subsystem 6200 is in fluidic communication with at least one channel, wherein the at least one channel is in fluidic communication with at least one exit port. In some embodiments, the at least one channel is formed by a space between spacer filter 2200 and either first outer shell 2100 or second outer shell 2300 of filtration assembly 2000. In some embodiments, the at least one channel is formed by the texture on the inner surface of first outer shell 2100 and/or second outer shell 2300. In some embodiments, the texture on First outer shell 2100 and/or second outer shell 2300 includes groves 7100 and bumps 7200.

In some embodiments, the at least one channel formed by the space between spacer filter 2200 and either first outer shell 2100 or second outer shell 2300 and/or the at least one channel formed by the texture on the inner surface of first outer shell 2100 and/or second outer shell 2300 facilitate the removal of waste material from the second subsystem by providing pathways for the waste materials to flow through, even when the second subsystem is collapsed or generally constricted due to the process (e.g., providing suction to exit port 3000) of waste material removal. In some embodiments, the at least one channel facilitates the removal of the waste material by providing a flow path for the waste material to exit port 3000 or drainage tube 3100 of lid 9100, which is inserted through exit port 3000. In some embodiments, the at least one channel facilitates the removal of the waste material by providing a flow path for the waste material to exit port 3000.

### Folded Filter

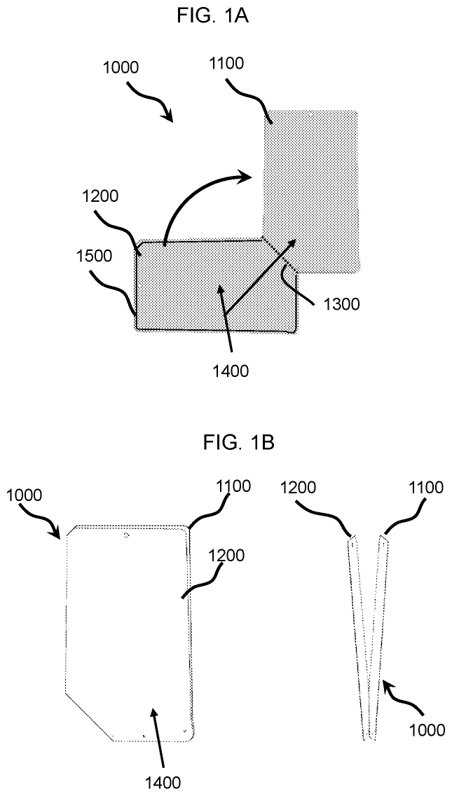

Filtration assembly 2000 of the disclosure includes folded filter 1000 (FIG. 1A). Folded Filter 1000 is made of a single sheet of filter material including first plurality of pores 1400. In some embodiments, first plurality of pores 1400 may have a diameter of about 30 μm to about 2000 μm (e.g., about 30 μm to about 2000 μm, about 35 μm to about 2000 μm, about 40 μm to about 2000 μm, about 50 μm to about 2000 μm, about 60 μm to about 2000 μm, about 70 μm to about 2000 μm, about 80 μm to about 2000 μm, about 90 μm to about 2000 μm, about 100 μm to about 2000 μm, about 110 μm to about 2000 μm, about 120 μm to about 2000 μm, about 130 μm to about 2000 μm, about 140 μm to about 2000 μm, about 150 μm to about 2000 μm, about 160 μm to about 2000 μm, about 170 μm to about 2000 μm, about 180 μm to about 2000 μm, about 190 μm to about 2000 μm, about 200 μm to about 2000 μm, about 250 μm to about 2000 μm, about 300 μm to about 2000 μm, about 350 μm to about 2000 μm, about 400 μm to about 2000 μm, about 450 μm to about 2000 μm, about 500 μm to about 2000 μm, about 600 μm to about 2000 μm, about 700 μm to about 2000 μm, about 800 μm to about 2000 μm, about 900 μm to about 2000 μm, about 1000 μm to about 2000 μm, or about 1500 μm to about 2000 μm, e.g., about 30 μm, about 35 μm, about 40 μm, about 50 μm 60 μm, about 70 μm, about, 74 μm, about 80 μm, about 90 μm, about 100 μm, about 110 μm, about 120 μm, about 130 μm, about 140 μm, about 150 μm, about 160 μm, about 170 μm, about 180 μm, about 190 μm, about 200 μm, about 210 μm, about 220 μm, about 230 μm, about 240 μm, about 250 μm, about 260 μm, about 270 μm, about 280 μm, about 290 μm, about and 300 μm, about 350 μm, about 400 μm, about 450 μm, about 500 μm, about 600 μm, about 700 μm, about 800 μm, about 900 μm, about 1000 μm, about 1500 μm, or about 2000 μm). In some embodiments, first plurality of pores 1400 may have a diameter of about 30 μm to about 200 μm (e.g., about 30 μm to about 200 μm, about 35 μm to about 200 μm, about 40 μm to about 200 μm, about 45 μm to about 200 μm, about 50 μm to about 200 μm, about 55 μm to about 200 μm, about 60 μm to about 200 μm, about 65 μm to about 200 μm, about 70 μm to about 200 μm, about 75 μm to about 200 μm, about 80 μm to about 200 μm, about 85 μm to about 200 μm, about 90 μm to about 200 μm, about 95 μm to about 200 μm, about 100 μm to about 200 μm, about 105 μm to about 200 μm, about 110 μm to about 200 μm, about 115 μm to about 200 μm, about 120 μm to about 200 μm, about 125 μm to about 200 μm, about 130 μm to about 200 μm, about 135 μm to about 200 μm, about 140 μm to about 200 μm, about 145 μm to about 200 μm, about 150 μm to about 200 μm, about 155 μm to about 200 μm, about 160 μm to about 200 μm, about 165 μm to about 200 μm, about 170 μm to about 200 μm, about 175 μm to about 200 μm, about 180 μm to about 200 μm, about 185 μm to about 200 μm, about 190 μm to about 200 μm, about 195 μm to about 200 μm, about 35 μm to about 195 μm, about 40 μm to about 190 μm, about 45 μm to about 185 μm, about 50 μm to about 180 μm, about 55 μm to about 175 μm, about 60 μm to about 170 μm, about 65 μm to about 165 μm, about 70 μm to about 160 μm, about 75 μm to about 155 μm, about 80 μm to about 150 μm, about 85 μm to about 145 μm, about 90 μm to about 140 μm, about 95 μm to about 135 μm, about 100 μm to about 130 μm, about 105 μm to about 125 μm, or about 110 μm to about 120 μm, e.g., about 30 μm, about 35 μm, about 40 μm, about 45 μm, about 50 μm, about 55 μm, about 60 μm, about 65 μm, about 70 μm, about 74 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 135 μm, about 140 μm, about 145 μm, about 150 μm, about 155 μm, about 160 μm, about 165 μm, about 170 μm, about 175 μm, about 180 μm, about 185 μm, about 190 μm, about 195 μm, or about 200 μm). In some embodiments, first plurality of pores 1400 may have a diameter of about 30 μm to about 100 μm (e.g., about 30 μm to about 100 μm, about 35 μm to about 100 μm, about 40 μm to about 100 μm, about 45 μm to about 100 μm, about 50 μm to about 100 μm, about 55 μm to about 100 μm, about 60 μm to about 100 μm, about 65 μm to about 100 μm, about 70 μm to about 100 μm, about 75 μm to about 100 μm, about 80 μm to about 100 μm, about 85 μm to about 100 μm, about 90 μm to about 100 μm, about 95 μm to about 100 μm, about 35 μm to about 95 μm, about 40 μm to about 90 μm, about 45 μm to about 85 μm, about 50 μm to about 80 μm, about 55 μm to about 75 μm, or about 60 μm to about 70, e.g., about 30 μm, about 35 μm, about 40 μm, about 45 μm, about 50 μm, about 55 μm, about 60 μm, about 65 μm, about 70 μm, about 74 μm, about 75 μm, about 80 μm, about 85 μm, about 90 μm, about 95 μm, or about 100 μm). In some embodiments, first plurality of pores 1400 may have a diameter of about 50 μm to about 200 μm. In some embodiments, first plurality of pores 1400 may have a diameter of about 74 μm. In some embodiments, the plurality of pores allows fluids and red blood cells to pass while retaining adipose tissue or a dehydrated adipose tissue. The single filter sheet is made of first filter segment 1100 and second filter segment 1200, which are joined at a corner by folding line of symmetry 1300 (FIG. 1A).

Upon folding the single sheet of the filter along folding line of symmetry 1300, the two filter segments, first filter segment 1100 and second filter segment 1200, align to generate folded filter 1000 (FIG. 1B). In some embodiments, the inside surface of first filter segment 1100 and the inside surface of second filter segment 1200 are joined, throughout the entire periphery of first filter segment 1100 and the entire periphery of second filter segment 1200, by double heat seal 1500, e.g., at the location shown in FIG. 1A, thereby forming an inner surface and an outer surface of folded filter 1000.

### Ports

Generally, the filtration assembly includes a plurality of ports for inputting an adipose tissue sample and for removing a dried or dehydrated adipose tissue after processing. The filtration assembly may also include a port dedicated to the removal of fluid(s) and unneeded cells (e.g., red blood cells) present in an unprocessed adipose tissue sample. The ports comprise at least one aperture that extends from the environment outside the filtration assembly into the interior of either first subsystem 6100 or second subsystem 6200. Each aperture has an airtight and watertight seal along the seams of the ports. As is discussed further below, in some embodiments, the ports are configured to be coupled to one or more connectors, conduits, port assemblies, adaptors, caps, or syringes.

Filtration assembly 2000 includes exit port 3000 and/or ports 4000 and/or 5000. In some embodiments, first subsystem 6100 includes two ports 4000 and 5000. In some embodiments, second subsystem 6200 includes one exit port 3000.

In some embodiments, port 5000 is an injection port. In some embodiments, adipose tissue is added to first subsystem 6100 of filtration assembly 2000 through port 5000. In some embodiments, port 4000 is connected to a container including a washing or rinsing solution, which allows washing or rinsing solution to enter first subsystem 6100. In some embodiments, exit port 3000 is operably coupled to a waste bag through which contents such as red blood cells, saline, mature adipocytes and the like are removed from second subsystem 6200.

Ports 4000 and 5000 may provide an access point for inserting various fluids, e.g., washing and rinsing fluids, and exit port 3000 may provide an access point for removing such fluids and effluent. Exit port 3000 and/or ports 4000 and/or 5000 can be sealed (e.g., with a valve). In some embodiments, exit port 3000 and/or ports 4000 and/or 5000 can be manually sealed with a clamp. In some embodiments, exit port 3000 and/or ports 4000 and/or 5000 can be sealed by a cap. In some embodiments, exit port 3000 and/or ports 4000 and/or 5000 include self-sealing valves, e.g., a deformable valve that provides for unidirectional flow of fluid or contents into, but not out of first subsystem 6100 or second subsystem 6200 of filtration assembly 2000. In some embodiments, the deformable valves provide for bi-directional flow. Deformable valves can be made from any deformable material known in the art, such as rubber, neoprene, silicone, polyurethane, or the like. In some embodiments, exit port 3000 and/or ports 4000 and/or 5000 comprise a Luer-activated valve. In some embodiments, exit port 3000 and/or ports 4000 and/or 5000 are located in the filtration assembly such that when the filtration assembly is held upright, exit port 3000 and/or ports 4000 and/or 5000 is positioned inferiorly such that fluids and effluent will egress with the assistance of gravitational forces, suction or pressure. Other ports may also be utilized with filtration assembly 2000, e.g., ports for venting, ports for adding materials or gases to filtration assembly 2000.

Exit port 3000 and/or ports 4000 and/or 5000 of filtration assembly 2000 can be configured to be directly or indirectly (e.g., via an adaptor) interconnected with or coupled to a syringe or catheter used to suction the adipose material from the source (e.g., a human body) such that the adipose tissue is directly transferred into filtration assembly 2000 aseptically. Similarly, a cannula or syringe may be attached to a port for aseptic transplantation of the partially disaggregated adipose tissue or fully disaggregated adipose tissue. Accordingly, in some embodiments, the ports on filtration assembly 2000 are configured to be directly or indirectly coupled with disposable or re-usable syringes. For example, in some embodiments, exit port 3000 and/or ports 4000 and/or 5000 are configured to be directly or indirectly coupled to a syringe with a volume of at least 1 cc (e.g., a syringe with a volume of at least 2 cc, at least 5 cc, at least 10 cc, at least 20 cc, at least 50 cc, at least 60 cc, at least 100 cc, or at least 250 cc), or the like. In some embodiments, the ports are configured to be directly or indirectly coupled to a syringe having a large bore tip, e.g., a Toomey syringe.

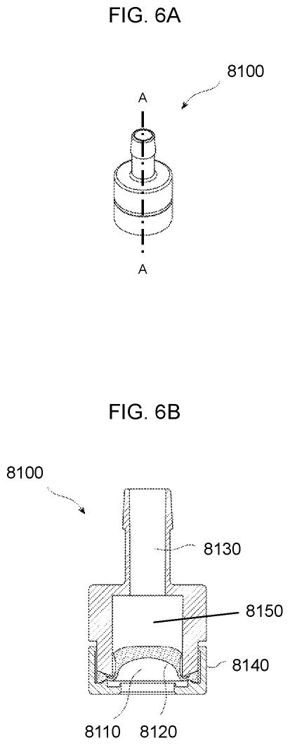

In some embodiments, filtration assembly 2000 comprises tissue access port assembly 8100 to facilitate aseptic delivery or removal of contents (e.g., adipose tissue) to and from first subsystem 6100. Exemplary tissue access port assembly 8100 is shown in greater detail in FIG. 6B. In some embodiments, tissue access port assembly 8100 includes a large bore cylindrical opening 8110. The large size of large bore cylindrical opening 8110 facilitates delivery and removal of tissue into first subsystem 6100. Deformable plastic valve 8120 can provide a barrier that prevents material from passing through large bore cylindrical opening 8110 into internal cavity 8150 of port assembly 8110 and further into narrow opening 8130. Deformable plastic valve 8120 can be configured to open and permit flow of contents through the large bore cylindrical opening 8110 into internal cavity 8150 of tissue access port assembly 8100, leading into the first subsystem of the filtration assembly by insertion of a cannula or a device comprising a lumen (e.g., a syringe tip) therein. Deformable plastic valve 8120 returns to a closed default state upon removal of the cannula or device comprising a lumen (e.g., a syringe tip) from deformable plastic valve 8120, thereby sealing tissue access port assembly 8100 and blocking the flow of any content (e.g., tissue or liquid) out of the internal cavity 8150 of tissue access port assembly 8100 when not in use. In some embodiments, the tissue access port assembly 8100 is configured to be connected to cap 8140 to provide an additional seal to the tissue access port when the port is not in use. In some embodiments, tissue access port assembly 8100 can be removable from filtration assembly 2000. In some embodiments, the tissue access port assembly is affixed to or joined to filtration assembly 2000, e.g. via an adhesive such as UV adhesive.

In some embodiments, tissue access port assembly 8100 can be configured to interconnect with an adaptor or connector. In some embodiments, the adaptor or connector is configured to join a syringe tip to a port of filtration assembly 2000. In some embodiments, tissue access port assembly is integral to, or removable from, port 5000. In some embodiments, the adaptor or connector comprises a Luer connector. In some embodiments, the adaptor or connector comprises a removal adaptor comprising a deformable valve.

In some embodiments, exit port 3000 and/or ports 4000 and/or 5000 of the filtration assembly 2000 can comprise, or be connected to, an assembly configured for more than one flow path, e.g., a Y connector or the like. In some embodiments, the Y connector is configured to allow obstruction of one or both flow paths, as desired, e.g., by clamping the individual lumens or flow paths of the Y connector. In some embodiments, the Y connector comprises a switch located at the junction of the common and individual flow paths of the Y connector that can be adjusted to allow for simultaneous flow through each individual flow path, one flow path, or to obstruct both flow paths, as desired.

In some embodiments, exit port 3000 and/or ports 4000 and/or 5000 of filtration assembly 2000 can be connected to conduits or tubing that allow for fluid communication to one or more external systems while maintaining a closed pathway.

In some embodiments, filtration assembly 2000 may comprise one or more exit ports (e.g., exit port 3000). In some embodiments, each of the one or more exit ports may provide an access point to second subsystem 6200 to facilitate removal of various fluids, e.g., washing and rinsing fluids, and effluent. In some embodiments, each of the one or more exit ports may be connected to an inlet of a branching connector external to filtration assembly 2000. In some embodiments, the branching connector includes at least 2 (e.g., at least 2, at least 3, at least 4, at least 5, or at least 6) inlets (e.g., that are connected to a corresponding exit port of filtration assembly 2000) and a single outlet. In some embodiments, the branching connector has 2 inlets and one outlet, (e.g., a Y connector).

### Filtration Assembly Holder

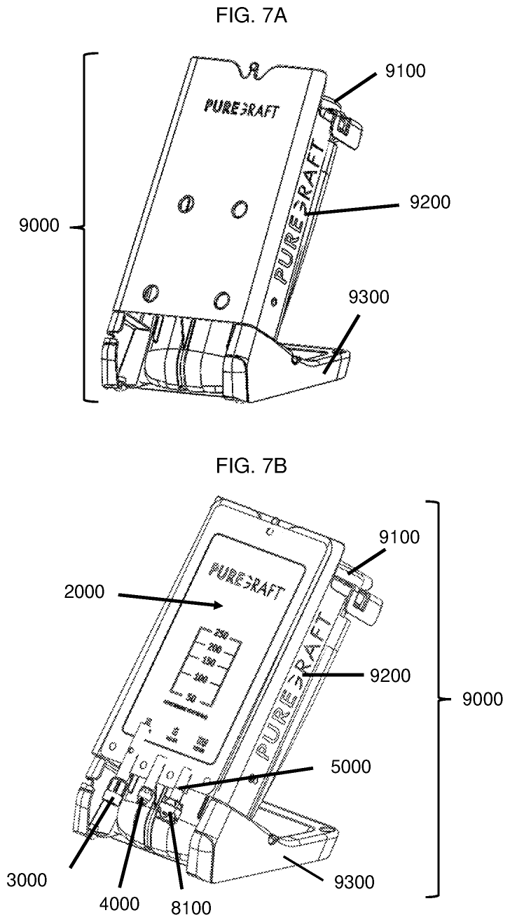

Filtration assembly 2000 of the present disclosure may be used (e.g., during preparation of adipose tissue grafts, preparation of dehydrated adipose tissue grafts, washing of adipose tissue, washing of dehydrated adipose tissue, removal of adipose tissue graft, or removal dehydrated adipose tissue grafts) with filtration assembly holder 9000. Filtration assembly 2000 may be internal to filtration assembly holder 9000 during preparation (e.g., injection of adipose tissue, washing of adipose tissue, and/or drying of adipose tissue) or on an external surface of filtration assembly holder 9000 during post-processing (e.g., drying of adipose tissue, removal of dehydrated adipose tissue graft, see FIG. 7B).

In some embodiments, filter assembly 2000 is oriented with exit port 3000, and/or ports 4000 and/or 5000 oriented upwards during use (e.g., during preparation of adipose tissue or dehydrated adipose tissue grafts) within filtration assembly holder 9000. In some embodiments, filter assembly 2000 is oriented with exit port 3000, and/or ports 4000 and/or 5000 oriented downwards during use (e.g., during removal of adipose tissue or dehydrated adipose tissue grafts) while attached to the external surface of filtration assembly holder 9000.

In some embodiments, filtration assembly holder 9000 may be attached to a waste container. In some embodiments, filtration assembly holder 9000 may be attached to a source of suction (e.g., vacuum) to facilitate removal of waste material from filtration assembly 2000.

In some embodiments, drainage tube 3100 may be attached to drainage channel 9130 of lid 9100 of filtration assembly holder 9000. In some embodiments, placement of filter assembly 2000 within holder 9000 requires insertion of drainage tube 3100 attached to drainage channel 9130 through exit port 3000 and into second subsystem 6200.

### Adipose Tissue

Some embodiments provided herein relate to methods of producing dried or dehydrated adipose tissue grafts that can be used directly in autologous transplantation procedures, e.g., autologous transplantation, or that can be supplemented with cells (e.g., adipose-derived regenerative cells, adipose-derived stem cells, or the like), additives or the like prior to transplantation.

The term adipose tissue, in some contexts, may refer to fat including the connective tissue that stores fat. Adipose tissue contains multiple regenerative cell types, including adipose-derived stem cells (“ADSCs”), endothelial progenitor and precursor cells, pericytes, macrophages, fibroblasts, lymphatic cells including lymphatic endothelial cells, etc., bound up by the connective tissue matrix. In some embodiments, a unit of adipose tissue is removed from a subject to generate an adipose tissue graft. In some embodiments, a unit of adipose tissue is a unit of unprocessed adipose tissue.

In some embodiments, one or more units of adipose tissue is/are removed from a subject. The adipose tissue used in the embodiments described herein can be obtained by any method known to a person of ordinary skill in the art. For example, adipose tissue can be removed from a subject by suction-assisted lipoplasty, ultrasound-assisted lipoplasty, excisional lipectomy, laser lipoplasty, water jet lipoplasty, or the like. In addition, the procedures may include a combination of such procedures, such as a combination of excisional lipectomy and suction-assisted lipoplasty. Preferably, the adipose tissue is collected in a manner that preserves the viability of the tissue and its cellular component and minimizes the likelihood of contamination of the collected material with potentially infectious organisms, such as bacteria and/or viruses. Thus, in preferred embodiments, the tissue extraction is performed in a sterile or aseptic manner to minimize contamination, e.g., in a closed sterile fluid/tissue pathway. In some embodiments, suction assisted lipoplasty is used to remove the adipose tissue from a subject, thereby providing a minimally invasive method of collecting tissue with reduced potential for cell or tissue damage that may be associated with other techniques, such as ultrasound assisted lipoplasty.

For suction-assisted lipoplastic procedures, adipose tissue is collected by insertion of a cannula into or near an adipose tissue depot present in the subject followed by aspiration of the adipose into a suction device. In one embodiment, a small cannula can be coupled to a syringe, and the adipose tissue can be aspirated using manual force. Using a syringe or other similar device can be used to harvest relatively moderate amounts of adipose tissue (e.g., from 0.1 ml to several hundred milliliters of adipose tissue). Procedures employing these relatively small devices have the advantage that the procedures can be performed with only local anesthesia, as opposed to general anesthesia. Larger volumes of adipose tissue above this range (e.g., greater than several hundred milliliters) may require general anesthesia at the discretion of the donor and the person performing the collection procedure. When larger volumes of adipose tissue are desired to be removed, relatively larger cannulas and automated suction devices can be employed in the procedure.

Excisional lipectomy procedures include, and are not limited to, procedures in which adipose tissue-containing tissue (e.g., skin) is removed by excision such as surgical dissection under direct or indirect visualization of the tissue being excised. In certain embodiments this may occur as an incidental part of the procedure; that is, where the primary purpose of the surgery is the removal of tissue (e.g., skin in bariatric or cosmetic surgery) and in which adipose tissue is removed along with the tissue of primary interest.

The amount of adipose tissue collected for use in the methods disclosed herein is dependent on a number of variables including, but not limited to, the body mass index of the donor, the availability of accessible adipose tissue harvest sites, concomitant and pre-existing medications and conditions (such as anticoagulant therapy), and the purpose for which the tissue is being collected. Engraftment of adipose tissue transplants has been shown to be cell dose-dependent with threshold effects. Thus, it is likely that the general principle that “more is better” will be applied within the limits set by other variables and that where feasible the harvest will collect as much tissue as possible.

In some embodiments, e.g., in embodiments in which the dried or dehydrated adipose tissue is used to make a supplemented adipose tissue graft, a unit of adipose tissue is divided into portions. The first portion of the adipose tissue is not disaggregated, and is either not processed at all, or rinsed or washed to obtain dried or dehydrated adipose tissue as described herein. The first portion of unprocessed, dried, or dehydrated adipose tissue can serve as the graft foundation that is supplemented with adipose-derived regenerative cells (e.g., a cell population that comprises adipose-derived stem cells, and/or endothelial cells and/or endothelial progenitor cells) present in the lipoaspirate or concentrated population of adipose-derived cells from the second portion. One portion can be processed, as is known the art, to release or liberate the adipose-derived regenerative cells (e.g., a cell population that comprises adipose-derived stem cells, and/or endothelial cells and/or endothelial progenitor cells) from the connective tissue matrix to obtain a lipoaspirate, or concentrated population of adipose-derived cells comprising regenerative cells or stem cells. In some embodiments, two different units of adipose tissue are collected, e.g., from the same or different regions of the subject, or from different subjects. One unit can be processed as described below to obtain lipoaspirate or a concentrated population of adipose-derived cells comprising regenerative cells or stem cells, and the other unit can serve as the foundation for the fat graft that is supplemented with adipose-derived regenerative cells (e.g., lipoaspirate and/or a cell population that comprises adipose-derived stem cells, and/or endothelial cells and/or endothelial progenitor cells). In one embodiment one or both of the units of adipose tissue may be cryopreserved such that delivery of the graft to the subject may be separated in time from harvest of the tissue. In one such embodiment, one or both units of tissue may be cryopreserved within filtration assembly 2000 of the present disclosure, in which first subsystem 6100 and second subsystem 6200 of filtration assembly 2000 are fabricated from materials that retain mechanical and structural integrity during the processes of cryopreservation, cryostorage, thawing, and subsequent use, as described herein. In another embodiment, the lipoaspirate or concentrated population of adipose-derived cells comprising regenerative cells including adipose-derived stem cells may be cryopreserved prior to use in fortification of the graft or implant as described herein.

In some embodiments, dried or dehydrated adipose tissue produced by methods described herein has from about 1% to about 95% of the liquid content (e.g., water, serum, or plasma) of an equivalent unit of unprocessed adipose tissue (e.g., 1% to 95%, 5% to 95%, 10% to 95%, 15% to 95%, 20% to 95%, 25% to 95%, 30% to 95%, 35% to 95%, 40% to 95%, 50% to 95%, 60% to 95%, 70% to 95%, 80% to 95%, 90% to 95%, or about 1%, 2%, 3%, 4%, 5%, 6%, 7%, 8%, 9%, about 10%, about 12%, about 15%, about 20%, about 25%, about 30%, about 35%, about 40%, about 45%, about 50%, about 55%, about 60%, about 65%, about 70%, about 75%, about 80%, about 85%, about 90%, or about 95% liquid content (e.g., by volume and/or weight) of an equivalent unit of unprocessed adipose tissue) or an equivalent unit of adipose tissue prepared by a conventional approach (e.g., centrifugation). In some embodiments, dried adipose tissue can have about 30% to about 90% (e.g., about 30% to about 90%, about 35% to about 90%, about 40% to about 90%, about 45% to about 90%, about 50% to about 90%, about 55% to about 90%, about 60% to about 90%, about 65% to about 90%, about 70% to about 90%, about 75% to about 90%, about 80% to about 90%, about 85% to about 90%, about 35% to about 85%, about 40% to about 80%, about 45% to about 75%, about 50% to about 70%, or about 55% to about 65, e.g., about 30%, about 35%, about 40%, about 45%, about 50%, about 55%, about 60%, about 65%, about 70%, about 75%, about 80%, about 85%, or about 90%)) less liquid content than an equivalent unit of unprocessed adipose tissue.

In some embodiments, dried or dehydrated adipose tissue described herein can contain a lower content or percentage of lipid and/or red or white blood cells as compared to an equivalent unit of unprocessed adipose tissue. In some embodiments, dried or dehydrated adipose tissue can have at less than about 1%, 2%, 3%, 4%, 5%, 6%, 7%, 8%, 9%, 10%, 12%, 15%, 20%, 25%, 30%, 35% 40%, 45%, 50%, 55%, 60%, 65%, 70%, 80%, 90%, 95% or 99% (or any % in between this range) (or any number in between this range) of the lipid content and/or red and/or white blood cells present in an equivalent unit of unprocessed adipose tissue and/or an equivalent unit of adipose tissue processed using a centrifugation method, e.g., wherein the excised tissue is spun in a fixed angle centrifuge or another conventional preparation technique. For example, in some embodiments, dried or dehydrated adipose tissue can contain less than about 75%, less than 80%, less than 85%, less than 90%, less than 95%, or less, or any % in between this range, of the number of the lipid content and/or red and/or white blood cells present in an equivalent unit of adipose tissue.

In some embodiments, the portion of processed adipose tissue (e.g., disaggregated lipoaspirate, or regenerative cell solution (e.g., solution of ADSC)), and/or a portion of unprocessed adipose tissue, dried adipose tissue, dehydrated adipose tissue, and/or a adipose tissue graft supplemented with adipose-derived regenerative cells described herein can be combined, supplemented, or mixed with additives such as other cells, tissue, tissue fragments, demineralized bone, or factors or agents, such as additives that lyse adipocytes and/or red blood cells. For example, in some embodiments, the portion of processed adipose tissue (e.g., dried or dehydrated adipose tissue), and/or a portion of unprocessed adipose tissue, and/or a adipose tissue graft supplemented with adipose-derived regenerative cells described herein can be combined, supplemented, or mixed with growth factor additives such as insulin or drugs such as members of the thiaglitazone family, antibiotics, biologically active or inert compounds, such as coagulases, cell-reaggregation inhibitors, resorbable plastic scaffolds, or other additive intended to enhance the delivery, efficacy, tolerability, or function of the population.

In some embodiments, the unprocessed adipose tissue, the dried adipose tissue, the dehydrated adipose tissue, and/or the supplemented adipose tissue grafts can be supplemented with one or more cellular differentiation agent additives, such as cytokines and growth factors. In some embodiments, the subject receiving the adipose tissue graft is also administered one or more cellular differentiation agents, such as cytokines and growth factors in a composition that is separate from the adipose tissue graft. For example, in some embodiments, the separate compositions may include angiogenic agents or other factors. In some embodiments, the unprocessed adipose tissue, the dried adipose tissue, the dehydrated adipose tissue, and/or supplemented adipose grafts described herein include an angiogenic factor(s) as an additive.

Angiogenic factors include those that can be produced or obtained from any suitable source. For example, the factors can be purified from their native source(s) or produced synthetically or by recombinant expression. The factors can be administered to subjects as a protein composition, in the form of an expression plasmid encoding the factors, or mixed in with the compositions disclosed herein. The construction of suitable expression plasmids is well known. Suitable vectors for constructing expression plasmids, include, for example, adenoviral vectors, retroviral vectors, adeno-associated viral vectors, RNA vectors, liposomes, cationic lipids, lentiviral vectors and transposons.

In some embodiments, the cells of the processed adipose tissue, e.g., regenerative cell solution, the cells of the unprocessed adipose tissue, the dried adipose tissue, the dehydrated adipose tissue, or the cells of the supplemented adipose tissue grafts described herein can also be modified by insertion of DNA or by placement in cell culture in such a way as to change or supplement the function of the compositions for derivation of a cosmetic, structural, or therapeutic purpose. For example, gene transfer techniques for stem cells are known in the art, as disclosed in Mosca, J. D., J. K. Hendricks, et al. (2000). “Mesenchymal stem cells as vehicles for gene delivery.” Clin Orthop (379 Suppl): S71-90, and may include viral transfection techniques, and more specifically, adeno-associated virus gene transfer techniques, as disclosed in Walther, W. and U. Stein (2000). “Viral vectors for gene transfer: a review of their use in the treatment of human diseases.” Drugs 60(2): 249-71, and Athanasopoulos, T., S. Fabb, et al. (2000). “Gene therapy vectors based on adeno-associated virus: characteristics and applications to acquired and inherited diseases (review).” Int J Mol Med 6(4): 363-75. Non-viral based techniques may also be performed as disclosed in Muramatsu, T., A. Nakamura, et al. (1998). “In vivo electroporation: a powerful and convenient means of nonviral gene transfer to tissues of living animals (Review).” Int J Mol Med 1(1): 55-62. In preferred embodiments, the cells of the processed tissue are not cultured. More preferably, the cells of the processed tissue are maintained within a closed, sterile system until their use, e.g., until they are either loaded onto a delivery device, combined with unprocessed or dried or dehydrated adipose tissue, or delivered directly into a subject.

In embodiments in which the adipose tissue grafts are administered to a subject other than the subject from which the cells and/or tissue were obtained, one or more immunosuppressive agents may be administered to the subject receiving the graft as an additive to reduce, and preferably inhibit, rejection of the transplant. Examples of immunosuppressive agents that can be used in the methods disclosed herein include agents that inhibit T-cell/B-cell costimulation pathways, such as agents that interfere with the coupling of T-cells and B-cells via the CTLA4 and B7 pathways, as disclosed in U.S. patent Pub. No. 20020182211. Other examples include cyclosporin, mycophenolate mofetil, rapamycin, and anti-thymocyte globulin.

The supplemented adipose tissue graft produced by the filtration assembly and/or methods of the present disclosure can be administered directly into the subject. Providing a closed system reduces the possibility of contamination of the material being administered to the subject. Thus, processing the adipose tissue and generating the supplemented adipose tissue graft while maintaining a closed system provides advantages over existing methods because the active cell population is more likely to be or remain sterile. In such an embodiment, the only time the supplemented adipose tissue graft is exposed to the external environment, or removed from the system, is when the cells or supplemented grafts are being withdrawn into an application device and being administered to a subject. In one embodiment, the application device can also be part of the closed system. Thus, in some embodiments, the supplemented adipose tissue grafts are not processed for culturing, or cryopreserved.

In some embodiments, at least a portion of the unprocessed tissue, the dried adipose tissue, the dehydrated adipose tissue, and/or supplemented adipose tissue graft is stored for later implantation/infusion. For example, the compositions disclosed herein (e.g., the unprocessed tissue, dried adipose tissue, dehydrated adipose tissue, concentrated cell adipose-derived cell populations and/or supplemented adipose tissue graft) can be divided into more than one aliquot or unit such that part of the composition is retained for later application while part is applied immediately to the subject.

At the end of processing, the supplemented adipose tissue graft can be loaded into a delivery device, such as a syringe, scaffold, absorbable capsule or implant, for placement into the recipient by, for example subcutaneous techniques. In other words, the supplemented fat graft or implant may be placed into the subject by any means known in the art, for example, they may be introduced into the dermis (subcutaneous), into tissue space, or into tissues (e.g., breast, buttocks, or the like), or other location. Particularly, the loading takes place while maintaining a closed system. Particular embodiments include placement by needle, catheter, or by direct surgical implantation in association with additives, such as a preformed matrix or absorbable breast-shaped capsules.

### Method of Use

A filtration assembly of the present disclosure may be used for producing dried or dehydrated adipose tissue grafts. The method includes obtaining a portion of unprocessed adipose tissue from a subject and providing the unprocessed adipose tissue to first subsystem 6100 of filtration assembly 2000 through port 5000. Folded filter 1000 selectively allows passage of fluids (e.g., water, free lipid, oil, lysed cells, or tumescent fluid) and red blood cells into second subsystem 6200 while retaining adipose tissue. Second subsystem 6200 can include spacer filter 2200 made from a material that is lipid-wicking and/or fluid-wicking (e.g., a meshwork design that draws fluid from the first subsystem), as described in further detail below. In some embodiments, spacer filter 2200 is made from a porous material, in which the second plurality of pores of the spacer filter 2200 are larger than the first plurality of pores 1400 of folded filter 1000. The fluids and red blood cells may be removed from second subsystem 6200 via exit port 3000 during processing of the adipose tissue using filtration assembly 2000.

A physiological wash solution (e.g., Lactated Ringer's solution, Hartmann's Solution, PLASMALTYE™, or saline) may be introduced to first subsystem 6100 via port 4000 or 5000, thereby rinsing or washing the adipose tissue within first subsystem 6100 and carrying further impurities into second subsystem 6200, where they are removed via exit port 3000. The physiological wash solution can be aseptically introduced into filtration assembly 2000. The washing step may be repeated one or more times. The wash solution may be warmed before being introduced to first subsystem 6100 of filtration assembly 2000 (e.g., the wash solution may be warmed to a temperature of between 35-40 °C).

In some embodiments, the wash solution is first introduced into second subsystem 6200 and allowed to pass through folded filter 1000 into first subsystem 6100, thereby coming in contact with the adipose tissue therein. In some embodiments, the wash solution is introduced into both first subsystem 6100 and second 6200 subsystem.

In some embodiments, after introduction of the wash solution to filtration assembly 2000, the adipose tissue and the wash solution are gently agitated (e.g., by inverting, squeezing, or rocking filtration assembly 2000) in order to facilitate the rinsing and/or separation of free lipid, red and white blood cells, and tumescent fluid from the adipose tissue in first subsystem 6100. In other embodiments, the adipose tissue within first subsystem 6100 is contacted with the wash solution, which is allowed to drain or be drawn from first subsystem 6100 of filtration assembly 2000 without agitation, e.g., by gravitational or wicking forces.

In some embodiments, the volume of wash solution used to rinse the adipose tissue can be greater than the volume of the adipose tissue. By way of example only, in some embodiments, greater than or equal to about 1 ml, 2 ml, 3 ml, 4 ml, 5 ml, 6 ml, 7 ml, 8 ml, 9 ml, 10 ml, 15 ml, 20 ml, 25 ml, 30 ml, 50 ml, 100 ml, 150 ml, 200 ml, 250 ml, 300 ml, 350 ml, 400 ml, 450 ml, 500 ml, 550 ml, 600 ml, 650 ml, 700 ml, 750 ml, 800 ml, 850 ml, 900 ml, 950 ml, 1000 ml 1100 ml, 1500 ml, 2000 ml or any amount in between these volumes, of wash solution can be introduced to filtration assembly 2000 and used to rinse/wash the adipose tissue.

During the washing or rinsing step, liquid in filtration assembly 2000, e.g., the wash solution, liquid present in the unprocessed adipose tissue (e.g., including free lipid, oil, blood cells, lysed cells from the adipose tissue, and blood components), and the like pass through first plurality of pores 1400 of folded filter 1000 between first subsystem 6100 and second subsystem 6200 of filtration assembly 2000. As such, second subsystem 6200 fills with liquid. Spacer filter 2200 within second subsystem 6200 can function to draw and retain fluid from the first subsystem into second subsystem 6200, e.g., acting as a wick. The movement of water, tumescent fluid, blood and free lipid out of first subsystem 6100, which contains the adipose tissue, dries and dehydrates the adipose tissue. The fluid is removed from second subsystem 6200 through exit port 3000. In some embodiments, fluid is removed from second subsystem 6200 using a pump or a vacuum or by gentle agitation of filtration assembly 2000 (e.g., by inverting, squeezing, or rocking filtration assembly 2000). In some embodiments, fluid is allowed to drain from exit port 3000, thereby exiting from second subsystem 6200.

In some embodiments, the steps of adding wash solution and removing contents from the chamber corresponding to second subsystem 6200 can be repeated at least 1 time (e.g., at least 2, at least 3, at least 4, at least 5, at least 6, at least 7, at least 8, at least 9, or at least 10 times) or more.

In some embodiments, removal of the wash or rinse solution dehydrates the adipose tissue by at least about 50% (e.g., about 50%, about 55%, about 60%, about 66.6%, about 70%, about 75%, about 80%, about 85%, or about 90%). In some embodiments, the rinsed adipose tissue is dehydrated by removal of liquid content in an amount of at least 66.6% that of the first portion of unprocessed adipose tissue prior to dehydration.

The dried or dehydrated adipose tissue can be removed (e.g., aseptically) via port 5000, from first subsystem 6100. The dried or dehydrated adipose tissue can then be administered to a subject directly (e.g., without further processing). In some embodiments, the dried or dehydrated adipose tissue is further processed before administration to a subject (e.g., by the addition of an additive, as described in further detail below).

### Kits

Also featured are kits containing filtration assembly 2000 of the disclosure, which includes folded filter 1000, first outer shell 2100, second outer shell 2300, exit port 3000, ports 4000 and 5000, and one or more additional components. The one or more additional components may include one or more of spacer filter 2200, drainage tube 3100 (which may be attached to exit port 3000), a holder (e.g., filtration assembly holder 9000, to secure filtration assembly 2000 during adipose tissue processing (e.g., washing/rinsing, filtration, and purification processes), a waste bag (e.g., to hold any waste and wash fluids that pass through filtration assembly 2000), a wash solution holding container (e.g., configured to hold a wash solution and/or to deliver the wash solution to filtration assembly 2000), and tubing to connect exit port 3000, and/or ports 4000 and/or 5000 of filtration assembly 2000 to any of the above mentioned additional components or other external components not supplied within the kit. The components of the kit may be supplied in a disassembled form or the components may be supplied in the kit in a fully assembled form.

A kit may include a package insert containing instructions related to the operation of filtration assembly 2000. For example, the package insert may describe a method for the production dried or dehydrated adipose tissue grafts using a filtration assembly of the present disclosure.

### EXAMPLES

The following examples are meant to illustrate the invention. They are not meant to limit the invention in any way.

### Example 1: Adipose Tissue Filtration Using a Filtration Assembly

This example demonstrates the use of filtration assembly 2000 of the disclosure (see, e.g., FIG. 2), which includes folded filter 1000 for the filtration of adipose tissue to produce dried or dehydrated adipose tissue grafts.

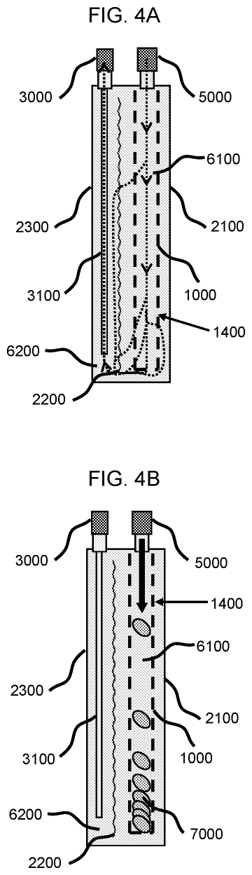

The method includes obtaining a portion of unprocessed adipose tissue and providing the unprocessed adipose tissue to first subsystem 6100 through injection port 5000 of filtration assembly 2000 (see, e.g., FIG. 3 and FIG. 4). The non-adipose tissue portion (e.g., fluids and red blood cells) of the provided material passes through folded filter 1000 and into second subsystem 6200, while the adipose tissue is retained. The non-adipose tissue portion flows through or around spacer filter 2200 within second subsystem 6200 (see, e.g., FIG. 4) and is removed from filtration assembly 2000 via drainage tube 3100 and exit port 3000. Removal of the non-adipose tissue portion may be assisted by an external force (e.g., vacuum, hand-pressure, etc.).

The adipose tissue retained in first subsystem 6100 can then be washed with ~150 mL of a wash solution (e.g., Lactated Ringer's solution), which can be added to filtration assembly 2000 via port 4000. Filtration assembly 2000 can be agitated to break up any clusters of adipose tissue and to enhance washing of the adipose tissue. The washing solution along with non-adipose tissue and other impurities (e.g., red blood cells and lysed cells) flow through folded filter 1000 to enter second subsystem 6200, which forms a separate discrete chamber. The contents that enter second subsystem 6200 can be removed from filtration assembly 2000 by exit port 3000 using drainage tube 3100. The washing step can be repeated once more with another volume of wash solution (e.g., up to ~150 mL of Lactated Ringer's solution), with or without agitation, and the wash solution and its contents are allowed to drain into second subsystem 6200. After removal of the wash solution from first subsystem 6100 into second subsystem 6200, the dehydrated adipose tissue that remains is ready to be removed from filtration assembly 2000 via port 5000 for further processing or to be used as an adipose tissue graft.

### Example 2: Use of Filtration Assembly with Filtration Assembly Holder

This example demonstrates the use of filtration assembly 2000 of the disclosure (see, e.g., FIG. 2), which includes folded filter 1000 within filtration assembly holder 9000, for the filtration of adipose tissue in a sterile closed system to prepare adipose tissue for use in as adipose tissue graft (e.g., as a dried or dehydrated adipose tissue graft).

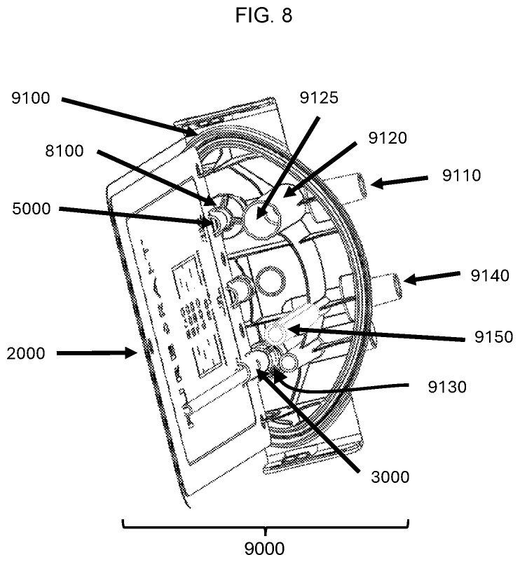

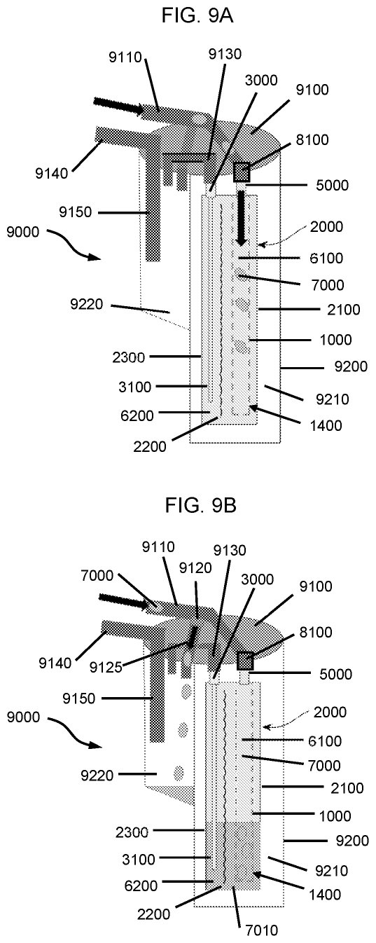

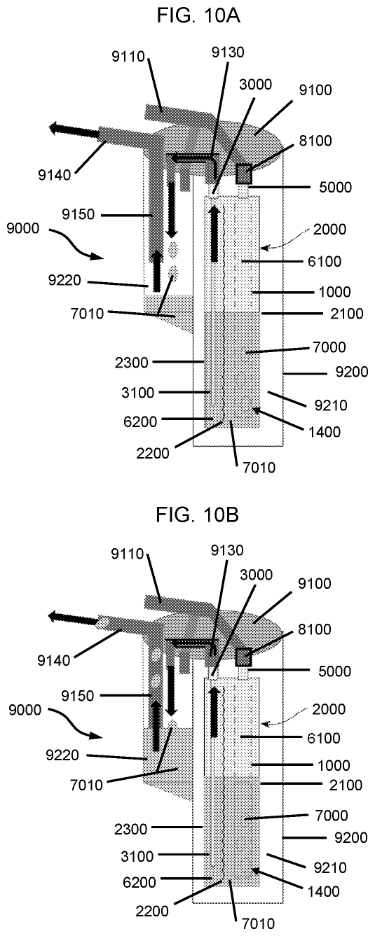

Filtration assembly 2000 is attached to lid 9100 of filtration assembly holder 9000 (e.g., see FIG. 8) and placed within filtration assembly chamber 9210 of body 9200 (e.g., see FIGS. 9 and 10). Briefly, filtration assembly 2000 is connected to Lid 9100 via access port assembly 8100 that is connected to port 5000. Lid 9100 is further attached to filtration assembly 2000 via exit port 3000 attached to drainage channel 9130.

As a first action, tubing is connected to vacuum connector 9140 and tissue inlet 9110. Filtration assembly holder 9000 is intended to be used with a vacuum or aspirator apparatus as a vacuum source. Tissue inlet 9110 is then connected, via the tubing, to a sterile source of adipose containing material (e.g., lipoaspirate). When the vacuum is initiated, filtration assembly holder 9000 is placed under a closed vacuum system, which initiates the introduction of adipose containing material into filtration assembly 2000. As the material flows into filtration assembly 2000, adipose tissue is trapped within first subsystem 6100 formed by folded filter 1000 (e.g., see FIG. 9A). Waste materials 7010 (e.g., wash solution, water, plasma, red blood cells, and/or oils) flow freely through first plurality of pores 1400 of folded filter 1000 and into second subsystem 6200.

As waste material 7010 fills second subsystem 6200, it is removed from second subsystem 6200 of filtration assembly 2000 via drainage tube 3100 and into drainage channel 9130, which is attached to exit port 3000, thereby flowing into waste chamber 9220 (e.g., see FIG. 10A). In some instances, when the internal pressure within filtration assembly holder 9000 reaches the lower bound of the safe operating range, bypass valve 9120 may open and provide drainage for incoming materials, via tissue inlet 9110, into bypass channel 9125, which is then deposited directly into waste chamber 9220 (e.g., see FIG. 9B). The internal pressure within filtration assembly holder 9000 may reach the lower bound of the safe operating range by improper vacuum control or if first plurality of pores 1400 of folded filter 1000 become substantially clogged with material (e.g., adipose tissue 7000), creating a blockage not allowing for equilibration of the tissue inlet and vacuum pressures. In this instance, bypass valve 9120 can be opened to relieve the pressure within filtration assembly 2000.

Upon reaching waste drainage tube 9150 within waste chamber 9220, waste material 7010 is evacuated out of waste chamber 9220 via waste drainage tube 9150 and vacuum connector 9140 (e.g., see FIG. 10B). Remaining adipose tissue 7000 within first subsystem 6100 may be further washed by introducing a washing solution via tissue inlet 9110. The wash solution, and any remaining waste material 7010, may then be removed from second subsystem 6200 after flowing through first plurality of pores 1400 of folded filter 1000, via drainage tube 3100 of lid 9100, through exit port 3000, and further via drainage channel 9130 into waste chamber 9220. After an appropriate number of wash cycles (e.g., at least one wash cycle) and drainage of filtration assembly 2000 to produce a dried or dehydrated adipose tissue graft, filtration assembly 2000 may be removed from filtration assembly holder 9000 and placed on the outer surface of filtration assembly holder 9000 for further processing or removal of the processed adipose tissue (e.g., dried or dehydrated adipose tissue graft; e.g., see FIG. 7B).

### Example 3: Providing a Subject with a Dried or Dehydrated Adipose Tissue Graft

A subject in need of or desiring a fat graft (e.g., in the context of a breast reconstructive surgery, breast augmentation, or buttocks augmentation) can be administered an adipose tissue graft prepared according to the methods described herein. A portion of adipose tissue can be extracted from the subject and a dried or dehydrated adipose tissue graft can be prepared using filtration assembly 2000, e.g., according to the methods described in Example 1 or Example 2.

If desired, the fat graft obtained by using filtration assembly 2000 can be enriched with adipose derived regenerative cells (ADRCs). The ADRCs can be obtained by any method described herein or known in the art (such as by use of the CELUTION® System). The ADRCs could be added to filtration assembly 2000 via port 5000. First subsystem 6100 is then washed with Lactated Ringer's or other suitable solution for 5 seconds or more. The system is agitated, e.g., for 15 seconds, and the flow liquid that has flowed through to second subsystem 6200 is removed through exit port 3000 via drainage tube 3100. The ADRC-supplemented dried or dehydrated adipose tissue graft may then be extracted from the first subsystem via port 5000 (e.g., into a syringe) and administered to the subject (e.g., by injection), as needed.

### Other Embodiments

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention.

The complete disclosures of all patents, patent applications including provisional patent applications, publications including patent publications and non-patent publications, and electronically available material cited herein are incorporated by reference.

Other embodiments are in the claims.

1. A filtration assembly for processing an adipose tissue, wherein the assembly comprises:
a) a first outer shell and a second outer shell that are sealingly joined to form a compartment within the assembly, wherein each of the first outer shell and/or the second outer shell comprises a surface;
b) a folded filter formed by a first segment folded onto a second segment that is located between the first outer shell and the second outer shell, wherein the folded filter forms a first subsystem within an interior of the folded filter and a second subsystem located between the first outer shell and the second outer shell on an exterior of the folded filter, wherein the folded filter further comprises a first plurality of pores, the second subsystem optionally further comprises a spacer filter; and the second subsystem is in fluid communication with at least one channel;
c) one or more ports in fluidic communication with the first subsystem; and
d) one or more exit ports in fluid communication with the second subsystem and/or the at least one channel;
wherein the one or more ports in communication with the first subsystem are configured to receive a material comprising the adipose tissue and to deliver the adipose tissue to the first subsystem, wherein the first plurality of pores are sized to retain adipose cells present in the adipose tissue, and wherein the at least one channel in the second subsystem is in fluid communication with the one or more exit ports configured to discharge the non-adipose tissue from the filtration assembly.

2. The filtration assembly of claim 1, wherein:
a) the first outer shell and the second outer shell are joined by a double heat seal;
b) the first segment and the second segment of the folded filter are joined by a double heat seal, thereby forming the interior of the folded filter;
c) the exterior of the folded filter is joined to the first outer shell and the second outer shell by a double heat seal;
d) the first plurality of pores has a pore size of from about 30 μm to about 2000 μm and/or the first plurality of pores is sized to allow passage of non-adipose tissue comprising one or more fluids and red blood cells present in the adipose tissue into the second subsystem, thereby removing fluid from the adipose tissue and producing a dehydrated adipose tissue, wherein, optionally, the one or more fluids comprise a tumescent fluid and/or one or more wash solutions;
e) the adipose tissue processed by the assembly comprises mature adipocytes, regenerative cells, stem cells, progenitor cells, and/or connective tissue; and/or
f) the assembly comprises the spacer filter, wherein the spacer filter has a second plurality of pores with a pore size of from about 300 μm to about 3000 μm and/or is free floating within the second subsystem.

3-8. (canceled)

9. The filtration assembly of claim 2, wherein the first plurality of pores has have a pore size of from about 30 μm to about 200 μm.

10. (canceled)

11. The filtration assembly of claim 2, wherein the pore size of the second plurality of pores of the spacer filter is about 800 μm.

12. (canceled)

13. The filtration assembly of claim 2, wherein the spacer filter comprises one or more ribs or struts or wherein the at least one channel is formed by the spacer filter and the first outer shell and/or the second outer shell of the assembly.

14-15. (canceled)

16. The filtration assembly of claim 1, wherein:
a) the at least one channel is formed by a texture on a surface of the first outer shell and/or the second outer shell of the assembly;
b) the at least one channel is configured to promote passage of one or more fluids and red blood cells to the one or more exit port; and/or
c) the one or more ports in fluidic communication with the first subsystem comprises a first port.

17. The filtration assembly of claim 16, wherein the texture on the surface of the first and/or second outer shell of the assembly comprises bumps, ridges, or grooves.

18-19. (canceled)

20. The filtration assembly of claim 16, wherein:
i) the first port is an injection port configured to releasably connect with an adapter, wherein the adapter is configured to releasably connect with a syringe barrel;
ii) the first port is a tissue access port assembly comprising a deformable plastic valve configured to seal the tissue access port assembly; wherein the deformable plastic valve is configured to facilitate delivery of material to the first subsystem or removal of material from the first subsystem upon insertion of a cannula therethrough;
iii) the first port comprises a deformable plastic valve; or
iv) the first port is configured to be attached to a cannula.

21. The filtration assembly of claim 20, wherein the syringe barrel is a catheter syringe barrel, wherein, optionally, the syringe has a volume of about 15 mL to about 100 mL.

22-24. (canceled)

25. The filtration assembly of claim 1, wherein a first exit port of the one or more exit ports is in communication with the second subsystem and is configured to discharge non-adipose tissue from the first exit port, wherein the first exit port is configured to inhibit the material from entering the first exit port, or wherein the assembly further comprises a second port of the one or more ports in fluidic communication with the first subsystem.

26. (canceled)

27. The filtration assembly of claim 25, wherein the one or more exit ports is connected to a branching connector.

28. A method of producing an adipose tissue graft comprising:
(a) introducing an adipose tissue into a first subsystem of a filtration assembly of claim 1 via the one or more ports in fluidic communication with the first subsystem; and
(b) removing a flow-through fluid that passes from the first subsystem to a second subsystem of the filtration assembly via the one or more exit ports, wherein the flow-through fluid comprises water, blood, free lipid, or any combination thereof, thereby producing the adipose tissue graft.

29. The method of claim 28, wherein:
i) the adipose tissue is obtained from a subject prior to step (a);
ii) the adipose tissue is a lipoaspirate, an unprocessed portion of adipose tissue, partially disaggregated adipose tissue, or fully disaggregated adipose tissue;
iii) prior to step (b), the filtration assembly is agitated to produce a partially or fully disaggregated adipose tissue within the first subsystem; and/or
iv) the method further comprises:
(c) introducing a wash solution to the first subsystem via the one or more ports in fluidic communication with the first subsystem, thereby rinsing the adipose tissue; and/or
(d) removing a waste fluid that passes from the first subsystem to a second subsystem of the filtration assembly via the one or more exit ports, wherein the waste fluid comprises water. the wash solution, blood, free lipid, or any combination thereof, thereby producing a dehydrated adipose tissue graft.

30-32. (canceled)

33. The method of claim 29, wherein:
the wash solution comprises Lactated Ringer's solution, Hartmann's Solution, and/or saline;
during step (c) the filtration assembly is agitated to produce a partially or fully disaggregated adipose tissue within the first subsystem;
steps (c)-(d) are repeated at least once; and/or
the dehydrated adipose tissue graft has a liquid content that is less than about 50% of the liquid content of the adipose tissue of step (a).

34-36. (canceled)

37. The method of claim 33, wherein the dehydrated adipose tissue graft has a liquid content that is less than about 33.3% or 25% of the liquid content of the adipose tissue of step (a).

38. (canceled)

39. A method of treating a subject comprising administering an adipose tissue graft prepared according to the method of claim 1 to the subject.

40. The method of claim 39, wherein:
a) the adipose tissue graft is a dehydrated adipose tissue graft;
b) the adipose tissue graft administered to the subject is aseptically removed from the filtration assembly prior to administration;
c) the adipose tissue graft is administered to an area in the body of the subject in need of treatment; and/or
d) the adipose tissue graft is administered to the subject during reconstructive surgery or augmentation.

41-43. (canceled)

44. The method of claim 40, wherein the reconstructive surgery or augmentation is breast reconstructive surgery or augmentation.

45. A kit comprising the filtration assembly of claim 1 and, optionally, one or more of:
(i) a filtration assembly holder;
(ii) a waste container;
(iii) a wash solution holding container; and
(iv) a squeegee.
